(19) United States
(12) Patent Application Publication
(10) Pub. No.: US 2021/0106469 A1
(43) Pub. Date: Apr. 15, 2021

(54) WOUND DRESSING

(71) Applicant: LOHMANN & RAUSCHER GMBH & CO. KG, NEUWIED (DE)

(72) Inventors: KIRSTEN DODEL, NEUWIED (DE); MARC POREMBA, DERNBACH (DE); ANDREAS WALGENBACH, BUCHHOLZ (DE); MARC SANDHOF, RENGSDORF (DE)

(21) Appl. No.: 16/965,319

(22) PCT Filed: Jan. 8, 2019
(86) PCT No.: PCT/EP2019/050277

§ 371 Date: Jul. 28, 2020

(30) Foreign Application Priority Data
Jan. 31, 2018 (DE) 10 2018 102 126.7

(51) Int. Cl. A61F 13/02; A61L 15/18; A61L 15/26

(52) U.S. Cl. CPC A61F 13/0213; A61L 15/18; A61F 13/0266; A61F 2013/00157; A61F 13/0223; A61L 15/26; A61F 13/0226

(57) ABSTRACT

The invention relates to a wound dressing comprising a carrier layer and an absorbent wound contact layer fixed to the carrier layer, which has wound contact surfaces spaced from one another by interstices in which the wound contact layer is not in contact with the wound, the wound contact layer being designed to be absorbent overall and in that the wound contact surfaces are connected to each other by connection regions of the wound contact layer.

The invention relates to a wound dressing comprising a carrier layer and an absorbent wound contact layer fixed to the carrier layer, which has wound contact surfaces spaced from one another by gaps in which the wound contact layer has no contact with the wound.

Such dressings are used to treat conventional wounds and burns. In this case exudate exiting the wound is taken up by the wound contact layer. In modern wound dressings, the wound contact layer may comprise a hydrogel or a silica gel, whereby the gel swells due to ingestion of liquid, but does not dissolve. The use of appropriate wound contact layers has the advantage that a gel may both accommodate or absorb, as well as release liquids, so as to adjust a desired moisture milieu in the area of the wound. This can promote the healing process.

Hydrogels which can be used as a wound contact layer are disclosed in WO 2015/059501 A1. The disclosure of this document with respect to the composition of hydrogels and additives for hydrogels is hereby incorporated by express reference into this specification.

In the EP 2 410 962 B1 it is proposed to improve the structural integrity of a hydrogel layer by means of a reinforcing layer. However, the wearing comfort of corresponding wound dressings is impaired by the reinforcing layer. The wearing comfort of wound dressings with wound contact layers having a hydrogel is in any case impaired by the swelling of the wound contact layer when liquids are taken up.

The wound dressing is usually fixed with an adhesive to the skin surrounding the wound. The adhesive may in this case be provided in a region of the wound surrounding the wound contact layer being on the side of the support layer facing the wound. In many cases, the carrier layer is fully coated with the adhesive. The adhesive also serves to fix the wound contact layer on the carrier layer.

The wearing comfort of the wound dressing just described with a wound contact layer comprising a hydrogel or a silica gel, according to WO 2015/059501 A1, is improved by the wound contact layer being formed by discrete hydrogel islands, which are mounted at a distance from each other on the carrier layer. Such wound dressings of the type mentioned above can then be adapted to the wound or the skin surrounding the wound by adopting a desired shape. This may result in a satisfactory wearing comfort.

When using appropriate wound dressings, however, it may happen that individual hydrogel islands detach from the carrier layer and contaminate the wound. According to WO 2015/059501A1, in view of this deficiency, an additional reinforcing layer can be incorporated into the individual hydrogel islands. Additionally or alternatively, a continuous absorption layer without intermediate spaces between individual wound contact surfaces can be applied to the end faces of the hydrogel islands facing away from the carrier layer. This, in turn, reduces the wearing comfort of these wound dressings. In addition, the production process for such wound dressings is complicated.

In view of the above problems in the prior art, the invention has for its object to provide a wound dressing which on the one hand offers a satisfactory wearing comfort and on the other hand is easy to produce.

According to the invention, this object is achieved by a further development of the known wound dressings, which is essentially characterized in that the wound contact surfaces which are spaced from one another by interstices in which the wound contact layer has no contact with the wound are connected to one another by connection regions of the wound contact layer.

In this way, a wound contact layer is obtained, which has an overall coherent or contiguous topology. This avoids that individual wound contact layer islands can be easily detached from the carrier layer. Due to the distance between the wound contact surfaces, the wound contact layer, even in the swollen state in which it has already absorbed exudate, has sufficient flexibility to adapt it to the shape of the wound or of the skin surrounding the wound. The contact with the wound takes place at least in the area of the wound contact surfaces. In this area, the wound exudate can be absorbed directly by the wound contact layer. However, the wound exudate can also penetrate into the spaces between the wound contact surfaces and penetrate from there via lateral boundary surfaces into the wound contact layer and be absorbed there.

In a preferred embodiment of the invention, the wound contact layer in at least one connection region has a smaller thickness in a direction perpendicular to the carrier layer than in the region of the contact surfaces. In this embodiment of the invention, the carrier layer in the area of the gaps is covered by the wound contact layer. However, through the lower thickness of the wound contact layer in this connection area a desired bending portion of the wound contact layer is provided which favors pliability of the wound contact layer and thus of the entire wound dressing. In this embodiment exudate penetrating into the interspaces between the wound contact surfaces can already be accommodated in the region of the interstices of the wound contact layer. The wound contact surfaces can be provided by a knob- or nap-shaped structure of the wound contact layer.

In addition or as an alternative to the embodiment of the invention just described, the wound contact layer in at least one connection region may have approximately the same thickness as in the region of the wound contact surfaces and also in contact with the wound in this connection region. In this case, the wound contact surfaces, which are separated from one another by the intermediate spaces, in which the wound contact layer is not in contact with the wound, are connected to one another by the connecting areas which may also come into contact with the wound, wherein the gaps, in which the side of the support layer facing the wound is exposed, also provide desired bending points of the entire wound dressing, which favor the flexibility of the wound dressing as a whole.

The interstices between individual wound contact surfaces with wound dressings according to the invention may be realized by wound contact layers which are at least partially concavely bounded. In the context of this invention, a topology of the wound contact layer is described by “concavely bounded” in cases where individual points on the edges of the wound contact layer are connected to each other at least in sections by straight lines running outside the wound contact layer. A concave boundary can also be provided by, for example, the wound contact layer as a whole being bounded in a bone or eight shape. In a preferred embodiment of the invention, at least two wound contact surfaces are at least partially approximately parallel to each other and preferably formed by approximately rectilinear wound contact strips. Between the wound contact strips a gap is formed, in which the wound-facing boundary surface of the carrier layer is not covered by the wound contact layer. This means that a concave boundary according to the invention can be provided both by rounded edges as well as by straight edge portions, as well as by combinations of rounded edge portions with straight edge portions.

The distance between the wound contact strips may be 0.5 mm or more. However, to provide sufficient absorption performance, the distance between the wound contact strips is desirably 30 mm or less, more preferably 20 mm or less, more preferably 10 mm or less. The spaced wound contact strips may enclose an acute angle of 20° or less.

In order to ensure the desired structural integrity of the wound contact layer while at the same time providing adequate flexibility of the wound contact layer, it has proved favorable if the wound contact strips emanate from a common wound contact bar, which preferably extends perpendicular to the wound contact strips, wherein the wound contact layer may also come into contact with the wound in the area of the wound contact bar. Additionally or alternatively, the wound contact bar can be made with a smaller thickness and form a gap between wound contact surfaces.

At least two wound contact strips can extend from the wound contact bar in opposite directions. In the context of the invention, it is also contemplated that wound contact strips and wound contact bar form a herringbone pattern with each other, in which the wound contact strips include an angle of less than 90°, but preferably more than 45° with the wound contact bar.

In addition or as an alternative to the embodiment of the invention just described, at least one wound contact strip may have at its one end a preferably curved connection region with a first adjacent wound contact strip approximately parallel or at an acute angle thereto and at its other end another, preferably also approximately curved executed connection region with a second adjacent wound contact strip disposed on the side facing away from the first adjacent wound contact strip. As mentioned above, the individual wound contact strips can be made approximately parallel to one another or at an acute angle.

As already explained above in connection with wound dressings according to the prior art, the wound contact layer can comprise or consist of a hydrogel and/or silica gel. Hydrogels which can be used in the context of the invention are described in WO 2015/059501 A1.

As with conventional wound dressings, an adhesive layer can be arranged between the carrier layer and the wound contact layer. In this case, the adhesive layer may also at least partially cover a region of the carrier layer that surrounds the wound contact layer and/or be applied in a grid or point like manner so as to enable the wound dressing to be attached to the skin surrounding the wound.

To avoid premature bonding of the adhesive layer, this layer and/or the wound contact layer can be covered by at least one cover strip detachably disposed thereon, such as a dehesively finished, for example Si-coated, waterproof film and/or silicone paper. In order to further improve the wearing comfort of wound dressings according to the invention, it can be provided that the carrier layer has or consists of a preferably transparent PU film. Such a polyurethane film can be made very thin, permeable to water vapor, and pliable, so as to allow optimum adaptation of the wound dressing to the shape of the wound and the skin surrounding the wound.

The application of wound dressings according to the invention can be facilitated if a support layer, in particular a support film, which is detachably attached to the side of the carrier layer facing away from the wound contact layer, is provided. In this case, the support film may preferably be connected via at least one film hinge with at least one cover strip. For application of wound dressings according to the invention, in the last-described embodiment of the invention the cover strip can first be removed from the wound dressing's boundary surface, then the wound dressing can be fixed on the wound or the skin surrounding the wound with the aid of the adhesive layer. Subsequently, the cover strip can be used as an aid to the detachment of the support layer or the support film so as to release the support layer from the carrier layer already fixed to the wound or the skin surrounding the wound.

In the following, the invention will be explained with reference to the drawing, to which reference is expressly made with regard to all details essential to the invention and details which are not further detailed in the description. In the drawing shows:

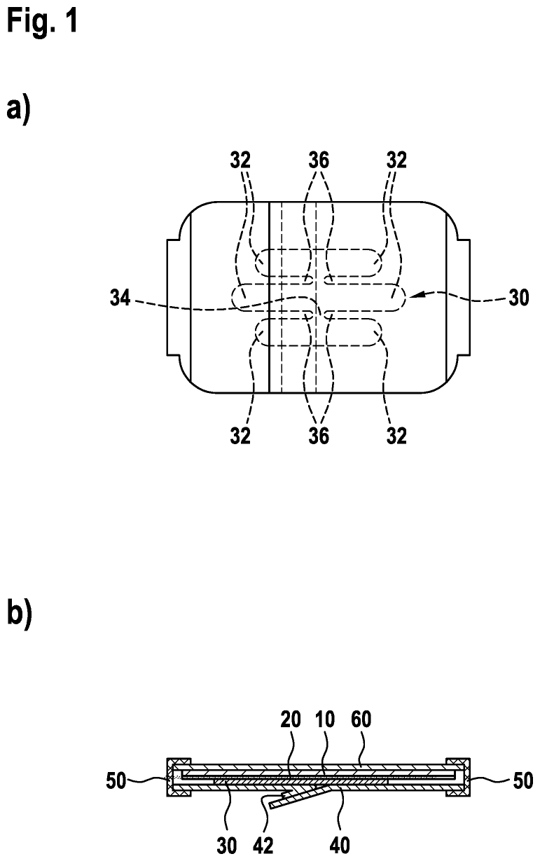

FIG. 1 a first embodiment of a wound dressing according to the invention,

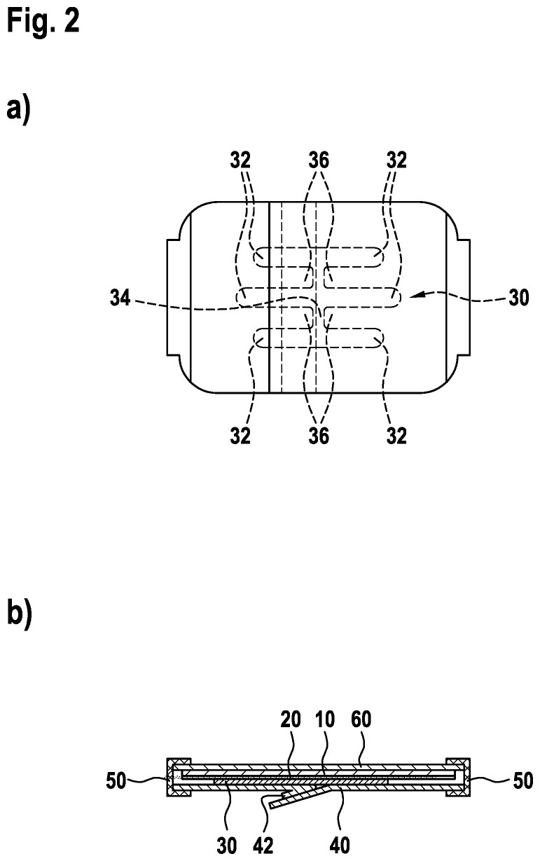

FIG. 2 a second embodiment of a wound dressing according to the invention,

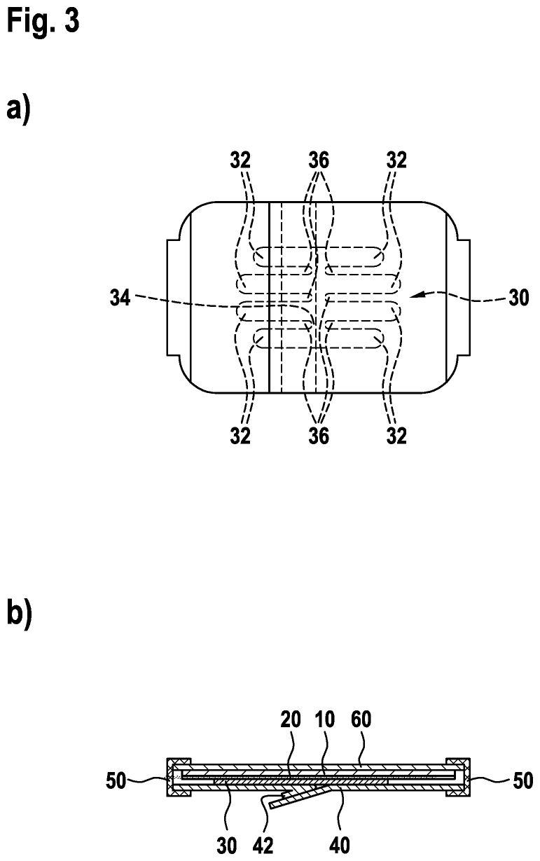

FIG. 3 a third embodiment of a wound dressing according to the invention and

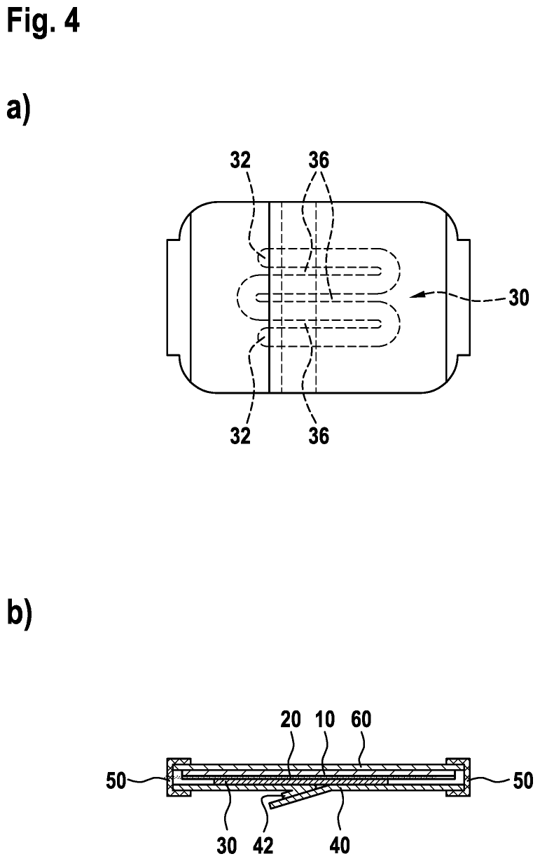

FIG. 4 A fourth embodiment of a wound dressing according to the invention.

In FIG. 1a) a top view of a wound dressing according to the invention is shown. FIG. 1b) shows a sectional view of the wound dressing shown in FIG. 1a). According to FIG. 1b), the wound dressing comprises a carrier layer 10, on which a wound contact layer 30 is fixed with the aid of an adhesive layer 20. The adhesive layer 20 circumscribes the wound contact layer 30 and thus makes it possible to fix the wound dressing in this peripheral adhesive area on the skin surrounding the wound. According to FIG. 1b), the wound contact layer 30 is covered by cover strips 40 and 42 on its side facing away from the carrier layer 10. The cover strips 40 and 42 are connected by hinges 50, which run around edges of the carrier layer 10, to a support film 60.

For application of the wound dressing illustrated in FIG. 1b), the cover strips 40 and 42 are released from the wound contact layer 30 or the surrounding adhesive region 12. The wound dressing can then be fixed with the aid of the adhesive layer 20 on the skin surrounding the wound. Following this, the cover strips 40 and 42 can be used as grasping aid with which the support film 60 connected thereto via the hinge joints 50 can be removed from the side of the carrier layer 10 facing away from the wound.

According to FIG. 1a), the wound contact layer 30 has a total of six wound contact strips 32, which are spaced apart from one another by gaps 36. In the area of the intermediate spaces 36, the carrier layer 10 is not covered by the wound contact layer 30. The wound contact strips 32 extend approximately parallel to each other and extend from a wound contact bar 34 in opposite directions approximately parallel to the edges of the carrier layer 10. The wound contact layer 30 according to the embodiment of the invention shown in FIG. 1 has in all areas a thickness of about 0.5 to 1.5 mm, in particular about 1 mm. In use it preferably lies completely against the wound on its side facing the wound.

The embodiment of the invention illustrated in FIG. 2 differs essentially from the embodiment shown in FIG. 1 in that the wound contact strips are 32 narrower and the interstices, in the region of which the substrate layer is not covered by the wound contact layer, are wider. As a result, a better flexibility of the wound dressing is achieved.

The embodiment of the invention illustrated in FIG. 3 essentially differs from the embodiment explained with reference to FIG. 1 in that a total of eight narrow contact strips extend in mutually opposite directions approximately parallel to one another starting from a wound contact bar 34. In this embodiment too, the flexibility of the wound dressing is increased by increasing the number of intermediate spaces in which the carrier layer is not covered by the wound contact layer. In addition, by increasing the number of strips in the in FIG. 3 embodiment, the absorption capacity can be increased.

In the embodiment of the invention shown in FIG. 4, the wound contact layer is made approximately meander-shaped. A total of four wound contact strips 32 are connected to one another via a total of three connection regions. The middle wound contact strips are connected at their one end via a curved connection region to a first adjacent wound contact strip extending approximately parallel thereto and at another end via a further connection region to a second adjacent wound contact strip arranged at a side of the at least one wound contact strip opposite to the first adjacent wound contact strip.

The invention is not limited to the embodiments explained with reference to the drawing. It is also thought of wound contact layers revolving spirally. Two, three or more wound contact bars can be provided, via which the wound contact strips can be connected to one another. It is essential that the wound contact layer as a whole constitutes a coherent structure which is fixed to the carrier layer and forms intermediate spaces in which no contact of the wound contact layer with the wound takes place.

1. Wound dressing with a carrier layer and an absorbent wound contact layer fixed to the carrier layer, which has wound contact surfaces spaced from one another by interstices in which the wound contact layer has no contact with the wound, the wound contact layer being designed to be absorbent overall, characterized in that the wound contact surfaces are connected to each other by connection regions of the wound contact layer.

2. Wound dressing according to claim 1, characterized in that the wound contact layer in at least one connecting region has a smaller thickness in a direction perpendicular to the carrier layer plane than in the region of the contact surfaces.

3. Wound dressing according to one of the preceding claims, characterized in that the wound contact layer has approximately the same thickness in at least one connecting region as in the region of the wound contact surface and also comes into contact with the wound in this region.

4. Wound dressing according to one of the preceding claims, characterized in that the wound contact layer is at least partially bounded concavely.

5. Wound dressing according to one of the preceding claims, characterized in that at least two wound contact surfaces are at least partially approximately parallel to each other and preferably formed by approximately rectilinear wound contact strips.

6. Wound dressing according to claim 5, characterized in that the distance between the wound contact strips is 0.5 mm or more, and/or 10 mm or less.

7. Wound dressing according to claim 5 or 6, characterized in that the wound contact strips emanate from a common wound contact bar, which preferably extends perpendicular to the wound contact strips.

8. Wound dressing according to claim 7, characterized in that at least two wound contact strips emanating from the wound contact bar extend in opposite directions.

9. Wound dressing according to one of claims 5 to 8, characterized in that at least one wound contact strip is connected at its one end via a preferably curved connection region to a first adjacent wound contact strip and at its other end via a further connection region to a second adjacent wound contact strip arranged at a side of the at least one wound contact strip opposite to the first adjacent wound contact strip.

10. Wound dressing according to one of the preceding claims, characterized in that the wound contact layer comprises or is a hydrogel and/or silica gel.

11. Wound dressing according to one of the preceding claims, characterized in that an adhesive layer is arranged between the carrier layer and the wound contact layer.

12. Wound dressing according to claim 11, characterized in that the adhesive layer and/or the wound contact layer is covered by at least one cover strip detachably mounted thereon and preferably made of silicone paper.

13. Wound dressing according to one of the preceding claims, characterized in that the carrier layer comprises or consists of a preferably transparent and/or water vapor-permeable PU film.

14. Wound dressing according to one of the preceding claims, characterized by a support layer, in particular a support film, detachably mounted on the side of the carrier layer facing away from the wound contact layer.

15. Wound dressing according to claim 14, characterized in that the support film is preferably connected via at least one film hinge with at least one cover strip.
